(19) United States
(12) Patent Application Publication
(10) Pub. No.: US 2011/0197497 A1
(43) Pub. Date: Aug. 18, 2011

(54) BROWN GREASE TREATMENT PROCESSES

(75) Inventor: Rong Jiang, Carbondale, IL (US)

(73) Assignee: MIDWEST ENERGY GROUP INC., Carbondale, IL (US)

(21) Appl. No.: 13/014,372

(22) Filed: Jan. 26, 2011

Related U.S. Application Data
(60) Provisional application No. 61/305,310, filed on Feb. 17, 2010.

(51) Int. Cl. C10L 1/00; F17D 1/18; A61L 9/00

(52) U.S. Cl. 44/307; 137/13; 422/5

(57) ABSTRACT

Provided herein is a process for rapidly reclaiming brown grease from a source of brown grease. In particular, the process comprises simultaneously transporting and heating the source of brown grease through a heated metal pipeline, and separating the lipid component comprising brown grease from other components in the source of brown grease. Also provided is a method for preparing a biodiesel solution from a lipid mixture comprising brown grease.

### CROSS-REFERENCE TO RELATED APPLICATION

This application claims priority to U.S. Provisional Application Ser. No. 61/305,310 filed on Feb. 17, 2010, which is hereby incorporated by reference in its entirety.

### GOVERNMENTAL RIGHTS

This invention was supported by funding from the Small Business Technology Transfer Program (STTR) (IIP-0711652) from the National Science Foundation. The United States Government has certain rights in this invention.

### FIELD OF THE INVENTION

The present invention relates to the reclamation of brown grease from sources of brown grease, the treatment of brown grease, and the use of brown grease as a feedstock for the production of biodiesel.

### BACKGROUND OF THE INVENTION

Biodiesel is an alternate fuel produced from natural, renewable biological materials. Typically, biodiesel comprises fatty acid methyl esters produced either through a transesterification reaction between the triglycerides in vegetable oils or animal fats with methanol or an esterification reaction between free fatty acids (FFAs) and methanol.

Brown grease recovered from grease trap waste and other aqueous waste media is an alternative feedstock for the production of biodiesel and other biofuels. Grease trap waste is collected from restaurants' grease interceptors or sewers and typically is disposed in wastewater treatment plants and landfill sites as environmental waste. The use of brown grease isolated from sewage as a feedstock will not only produce carbon-neutral biofuels but also reduce environmental pollutions. A recent Department of Energy study estimated that each year about 13 pounds of brown grease is generated per capita in the United States (http://www.nrel.gov/docs/fy99osti/26141.pdf). If this brown grease was recovered and used as feedstock, it could produce about 500 million gallons of biodiesel annually.

Brown grease needs to be reclaimed from grease trap waste or other aqueous waste media before it can be utilized as a raw material for biodiesel production or directly as a low-grade bunker fuel to replace coal. While there have been numerous attempts to recover or reclaim brown grease, many are not efficient or effective. A common problem for many of these methods is that brown grease tends to solidify at temperatures around 5-10° C. As a consequence, brown grease or grease trap waste tends to clog the pipelines used in the apparatuses for brown grease extraction. Although some methods may utilize a heater to melt or keep brown grease in a liquid phase, grease trap waste could clog the pipelines before it is fed into a heating chamber.

Additionally, most heating chambers for melting brown grease utilize a heat exchanger circulated with a hot liquid or an electric heating coil. For example, a heat exchanger is placed inside a tank in the presence of grease trap waste. A hot liquid (e.g., steam or hot water) circulating through the heat exchanger will melt brown grease that is in the close proximity to the surface of the heat exchanger. Due to small contact surface areas between brown grease and the heat exchanger, it is not a surprise that it will take a long time, typically over 12 hours to completely melt brown grease in 10,000 gallons of restaurant grease trap waste. Equal important, when grease trap waste is pumped through tanks and pipelines, detergents such as hand soaps in wastewater usually cause significant emulsions in the wastewater stream—making it difficult to separate brown grease from wastewater and solid components. This emulsion problem as well as the slow-melting challenge has made many methods not practical for processing a large quantity of sewer grease trap waste in a wastewater treatment plant. For instance, the Metropolitan Sewer District of St. Louis (St. Louis, Mo.) accepts over 100,000 gallons of wastewater collected from restaurant grease traps each day. As a result, a brown grease reclamation system that can process 10,000 gallons of restaurant wastewater in one hour is highly desirable for the practical reclamation of brown grease in a wastewater treatment plant. Given that the population of the greater St. Louis area is only about 2.9 million, wastewater treatment plants in other larger cities like Los Angeles, Calif. and New York City, N.Y., actually accept much more wastewater every day from restaurants. Therefore, a method and a system for rapid reclamation of brown grease are needed for practical applications in high yields.

Moreover, depending on the length of time inside a grease trap tank, triglyceride molecules of vegetable oil and animal fats become partially or completely hydrolyzed by water into FFAs, glycerol, monoglycerides, and diglycerides. As a result, brown grease recovered from grease trap waste usually contains a significant amount of FFAs, ranging from about 15 wt % to 100 wt %.

Numerous waste materials such as food waste, coffee residues, dirt and debris from restaurant floors, detergents/soaps/surfactants from dish washers and washing basins, human/animal waste, and microbes can be found inside a grease trap tank. Under anaerobic or aerobic conditions, a fermentation process by microbial systems can generate hydrogen sulfide and other volatile sulfur derivatives. Prolonged exposure to a large amount of hydrogen sulfide and volatile sulfur compounds could be detrimental to human health. As a result, strong odors have become a serious environment and health problem for separating and recovering brown grease from grease trap waste. Odor is also a problem for sewer brown grease itself due to a ppm level recognition threshold of many sulfur molecules and smell-producing bacterial contaminants. Consequently, a biodiesel plant using sewer brown grease as feedstock will face air emission issues. As a result, a deodorizing step is needed if sewer brown grease is used for biodiesel or other biofuel production at a commercial scale.

Brown grease also contains non-volatile sulfur chemicals such as sodium laureth sulfate and sodium dodecylbenzenesulfonate, which are common ingredients in hand soaps and detergents used in restaurants and households. As a result, it is not a surprise that sewer brown grease has a very high total sulfur value—typically well above 10,000 ppm. However, such sulfur materials must be removed if brown grease is used as a raw material for biofuel production. The U.S. Environmental Protection Agency has mandated the allowable sulfur content in ultra-low sulfur diesel to be 15 ppm or lower. Consequently, the biodiesel standard in the U.S. (ASTM D 6751-09 S15) has limited the total sulfur in B100 biodiesel to be no more than 15 ppm. Reduction of sulfur from 10,000 ppm in brown grease to 15 ppm in biodiesel may be a technological challenge for a biofuel producer.

Another problem associated with biodiesel produced from sewer brown grease is that, because brown grease contains a significant amount of FFAs, crude biodiesel product produced by an esterification reaction typically contains trace amounts of FFAs. Although esterification reactions can convert the majority of the FFAs into esters, a small amount of FFAs still remain in the esterification reaction product. This is because the esterification reaction involves an equilibrium between FFAs, methanol and fatty acid methyl esters and water. The conversion of 99% FFAs into methyl esters may lead to an acid number of the crude biodiesel to be around 5 mg KOH/g, which is above the acid number limit of 0.50 mg KOH/g established in ASTM D 6751-09. FFAs can initiate engine corrosion and affect human or animal health by emission of hazardous acrolein into the environment. As a consequence, residual FFAs in the esterification reaction product must be removed from crude biodiesel in order for the biodiesel to meet the ASTM specifications.

Thus, there is still a need for processes for (1) the rapid reclamation of brown grease from sewer brown grease or restaurant waste water, (2) deodorizing brown grease or sources of brown grease, (3) producing biodiesel from brown grease, and (4) reducing the levels of sulfur and FFAs in biodiesel produced from brown grease.

### SUMMARY OF THE INVENTION

One aspect of the disclosure provides a process for deodorizing a grease waste mixture. The process comprises contacting the grease waste mixture with a source of steam at a temperature from about 100° C. to about 160° C., an oxidizing agent, or a combination thereof, such that the grease waste mixture has a substantially reduced odor.

Another aspect of the disclosure encompasses a method for simultaneously transporting and heating a source of brown grease. The method comprises passing the source of brown grease through a metal pipeline that is heated with a flame to a temperature from about 25° C. to about 95° C. such that the source of brown grease has a dynamic viscosity from about 1.002×10⁻³ NS/m² to about 3.15×10⁻⁴ NS/m².

A further aspect of the disclosure provides a process for reclaiming brown grease from a source of brown grease, which comprises a lipid component, an aqueous component, and a solid component. The process comprises passing the source of brown grease through a metal pipeline that is heated with a flame to a temperature from about 25° C. to about 95° C. such that the source of brown grease has a dynamic viscosity from about 1.002×10⁻³ NS/m² to about 3.15×10⁻⁴ NS/m². The process further comprises separating the lipid component from the aqueous and solid components in the source of brown grease, wherein the separated lipid component comprises the reclaimed brown grease.

Still another aspect of the disclosure encompasses a process for reducing the levels of sulfur and free fatty acids in a crude biodiesel solution. The process comprises contacting the crude biodiesel solution with an insoluble base to form a treated biodiesel solution comprising at least one sulfur-insoluble base adduct and at least one free fatty acid-insoluble based adduct. The process further comprises removing the sulfur-insoluble and free fatty acid-insoluble base adducts from the treated biodiesel solution to generate a biodiesel solution having reduced levels of sulfur and free fatty acids.

Yet another aspect of the disclosure provides a process for preparing a biodiesel solution from a lipid mixture comprising brown grease. The process comprises contacting the lipid mixture comprising brown grease with an alcohol in the presence of an esterification catalyst to form a crude biodiesel solution. The process further comprises contacting the crude biodiesel solution with an insoluble metal oxide, an insoluble metal hydroxide, an insoluble metal silicate, or a combination thereof to form the biodiesel solution.

Other aspects and features of the processes disclosed herein are described in more detail below.

### DETAILED DESCRIPTION OF THE INVENTION

Provided herein are processes for reclaiming brown grease from sources of brown grease, and processes for preparing biodiesel from brown grease. In particular, processes are provided recovering or reclaiming brown grease from sources of brown grease (i.e., grease trap waste or grease-containing wastewater). Also provided are processes for reducing odors in brown grease or sources of brown grease, and processes for removing sulfur and FFA contaminants from a solution of crude biodiesel. Processes are also provided for preparing a biodiesel solution from a lipid mixture comprising brown grease. Thus, the processes disclosed herein facilitate rapid and efficient recovery of brown grease from sources of brown grease and address some of the problems associated with the use of brown grease as a feedstock for the production of biodiesel that meets ASTM standards.

(I) Process for Reclaiming Brown Grease from a Source of Brown Grease

One aspect of the disclosure provides a process for reclaiming or isolating brown grease from a source of brown grease. In particular, the process disclosed herein provides a means for the rapid recovery or reclamation of brown grease from sources of brown grease. The source of brown grease comprises a lipid component, which comprises the brown grease, as well as aqueous and solid components.

Typically, a vacuum truck loaded with waste collected from restaurants' grease traps brings the source of brown grease to the site of brown grease recovery. From an economic point of view, it is advantageous to station a brown grease extraction system in a wastewater treatment plant or a landfill site for in situ isolation of brown grease. In many regions, wastewater comprising fats, oils, and grease (i.e., FOG) is required by laws to be disposed at a wastewater treatment plant or a landfill site. Thus, after reclamation or isolation of the brown grease, the wastewater and solid components can be returned back to the wastewater treatment plant or a landfill company for conventional decontamination treatments.

In most instances, a connection pipe transports the source of brown grease from the vacuum truck into a storage tank or directly to a heating tank. The heating tank is usually equipped with a heat exchanger and/or jacketed by a heating element or a hot liquid. A hot liquid, usually hot water or steam, circulates through the heat exchanger to melt FOG inside the heating tank. Alternatively, electric heating coils may be placed inside the heating tank for melting FOG in the source of brown grease.

Frequently, and particularly under cold weather conditions, the connection pipe for transporting the source of brown grease from the vacuum truck to the holding or heating tank may get clogged with solidified FOG before the source of brown grease reaches the tank. Once inside the heating tank, the heat exchanger or electric heating coils inside the heating tank are able to melt solidified FOG that is close proximity to the heat exchanger or electric heating coils. However, the transfer of heat from the surface of the heat exchanger pipes or electric heating coil to other areas inside the tank is quite slow. Thus, raising the temperature of 10,000 gallons of grease trap waste or grease-containing wastewater from ambient temperature to 65° C. or higher using a heat exchanger or heating coils in the heating tank takes many hours—which makes it practically incapable of handling a large volume of the source of brown grease accepted each day in many wastewater treatment plants.

The process disclosed herein, therefore, comprises simultaneously transporting and heating the source of brown grease by passing the source of brown grease through a metal pipeline that is heated to a temperature from about 25° C. to about 95° C., such that the source of brown grease has a dynamic viscosity from about 1.002×10⁻³ NS/m² to about 3.15×10⁻⁴ NS/m². Advantageously, heating the source of brown grease while being transported through the metal pipeline facilitates the reclamation process because the source of brown grease may be rapidly heated to the desired temperature by virtue of the high surface to volume ratio of pipeline to the source of brown grease. The reclamation process further comprises separating the lipid component from the aqueous and solid components of the source of brown grease, wherein the isolated lipid component comprises the reclaimed brown grease. The reclamation process may further comprise centrifuging the isolated lipid component to de-emulsify and remove aqueous liquid from the lipid component comprising the reclaimed brown grease.

### (a) Source of Brown Grease

In general, the source of brown grease refers to “grease trap waste” or “grease-containing wastewater.” “Grease trap waste” refers to the material removed from grease traps (also known as grease interceptors, or grease recovery devices). Grease traps are plumbing devices designed to intercept most FOGs and solids before they can enter a wastewater disposal system. “Grease-containing wastewater” refers to any wastewater that contains FOGs. The source of the wastewater may be residential, commercial, industrial, agricultural, and so forth. For example, the grease-containing wastewater may be from residential households, rendering plants, and/or meat/poultry processing plants.

Each of the sources of brown grease, i.e., grease trap waste or grease-containing wastewater, typically comprise a grease or lipid component, an aqueous component, and a solid component. The percentages of the different components can and will vary depending upon the source of the material. The various components of the source of brown grease may further comprise waste materials such as dirt, debris, food waste, solid waste, soap detergents, microbial systems, and the like. The grease or lipid component of the source of brown grease may be completely or partially hydrolyzed into FFAs, glycerol, mono-glycerides, di-glycerides, and combinations thereof.

The source of brown grease may be pre-treated before being introduced into the heated metal pipeline. For example, particulate matter in the source of brown grease may be removed by filtration prior to be being introduced into the heated metal pipeline. That is, the source of brown grease may be passed through a screen, a mesh, or a filter prior to being introduced into the metal pipeline. In embodiments in which the source of brown grease is grease trap waste, the grease trap waste may be partially de-watered prior to being introduced into the metal pipeline. One skilled in the art will appreciate that depending on the nature of the source of brown grease, various pretreatments may be adopted to facilitate the transport and, ultimately, the separation of the lipid component comprising brown grease from the other components in the source of brown grease.

### (b) Transporting and Heating the Source of Brown Grease

The method comprises passing the source of brown grease through a metal pipeline that is heated to a temperature from about 25° C. to about 95° C. such that the source of brown grease has a dynamic viscosity from about 1.002×10⁻³ NS/m² to about 3.15×10⁻⁴ NS/m². Passing the source of brown grease through a heated metal pipeline quickly raises the temperature of the source of brown grease to about 25° C. to about 95° C. such that the source of brown grease is in a liquid state and cannot clog the pipeline with solidified FOG.

The means of passing the source of brown grease through the heated metal pipeline may vary. In one embodiment, the source of brown grease may be passed through the heated metal pipeline by gravity means. In another embodiment, the source of brown grease may be passed through the heated metal pipeline by pumping means.

The source of brown grease may be transported through the heated metal pipeline from various sources and/or tanks. For example, the source of brown grease may be transported from a vacuum truck to a holding tank, a heating tank, a separating tank, or a heating/separating tank. Alternatively, the source of brown grease may be transported from a holding tank to a heating tank, a separating tank, or a heating/separating tank. Once the source of brown grease is transferred to a heating and/or separating tank, the temperature of the source of brown grease may be maintained at about 25° C. to about 95° C.

The metal pipeline used for transporting the source of brown grease may be constructed of a variety of metallic materials. Without limiting the scope of the invention, examples of suitable metals include iron, copper, tin, lead, aluminum, magnesium, metal oxides, metal alloys like steel, stainless steel, cast iron, tool steel, alloy steel, derivatives, and combinations thereof.

The inner dimension of the metal pipeline can and will vary. In various embodiments, the inner dimension of the metal pipeline may range from about 200 inches to about 0.01 inch, from about 100 inches to about 0.1 inch, from about 24 inches to about 0.2 inch, from about 10 inches to about 0.5 inch, or from about 5 inches to about 1 inch. In one embodiment, the inner dimension of the metal pipeline may be about 4 inches. In another embodiment, the inner dimension of the metal pipeline may be about 3 inches.

The metal pipeline may also comprise a rise or a “fin,” which is a piece of metal that is not utilized for the conveyance of the source of brown grease can be used an appendage extending from the external body of the pipeline for increasing the contact area between the pipeline and the flame, when the source of heat is provide by a flame. The surface of the pipeline may also be coated with other materials to improve its heat conductivity.

The pipeline may be arranged in multiple dimensions. For instance, the pipeline may be stacked in three dimensions for improved heating efficacy.

The source of heat used to heat the metal pipeline may vary. In some embodiments, the source of heat may be provided by an electrical heating element wrapped around or in close contact with the metal pipeline. In another embodiment, the source of heat may be provided by a heat exchanger comprising circulating hot liquid that is wrapped around, jacketed, or in close contact with the metal pipeline. In an exemplary embodiment, the source of heat may be provided by a flame. A variety of flammable materials may be used for generating the flame. Non-limiting examples of suitable flammable materials include propane, diesel, biodiesel, brown grease, ethanol, butanol, methanol, coal, bunker fuel, waste, petroleum, gasoline, liquefied petroleum gas, woods, biomass, combinations and thereof.

In general, the distance and/or intensity of the flame will be adjusted such that the metal pipeline and the source of brown grease within the pipeline is heated to a temperature from about 25° C. to about 95° C. Preferentially, the metal pipeline is heated by the flame along the entire length of the pipeline or the three dimensional stack of pipeline.

In general, the metal pipeline and the source of brown grease will be heated to a temperature ranging from about 25° C. to about 95° C. In various embodiments, the temperature may range from about 25° C. to about 40° C., from about 40° C. to about 50° C., from about 50° C. to about 60° C., from about 60° C. to about 70° C., from about 70° C. to about 80° C., or from about 80° C. to about 90° C. The heating typically occurs at about 1 atmosphere.

The heated source of brown grease typically has a dynamic viscosity ranging from about 1.002×10⁻³ NS/m² to about 3.15×10⁻⁴ NS/m². In certain embodiments, the dynamic viscosity may range from about 1.002×10⁻³ NS/m² to 3.55×10⁻⁴ NS/m², or from 7.98×10⁻⁴ NS/m² to about 4.04×10⁻⁴ NS/m². In one embodiment, the dynamic viscosity may be about 4.67×10⁻⁴ NS/m².

In general, the final temperature and dynamic viscosity of the source of brown grease may affect the yield and purity of the recovered brown grease. For example, a low temperature and a high dynamic viscosity value generally lead to a low recovery of brown grease. For example, in embodiments in which the source of brown grease is grease-containing wastewater and the temperature of heating is low, there may be a low recovery of brown grease. In this situation, the low recovery of brown grease may lead to unacceptably high levels of FOG in the discharged wastewater. High FOG wastewater streams may clog the sewer pipelines and high levels of FOG may be problematic for microbial systems used in wastewater treatment plants for decontaminating the wastewater.

### (c) Optional Non-Polar Solvent

In some embodiments, the source of brown grease may be heated in the presence of a non-polar solvent. The non-polar solvent may be added to the metal pipeline, the heating/separating tank, or both. Non-limiting examples of suitable non-polar solvents include alkanes, substituted alkanes, cycloalkanes, acylglycerols, and combinations thereof. Suitable alkanes or cycloalkanes include hexanes, heptanes, pentane, octane, cyclohexane, and derivatives thereof. Suitable acylglycerols include glyceride mixtures such as vegetable oils (e.g., soybean oil) and yellow grease.

The amount of non-polar solvent combined with the source of brown grease can and will vary. In general, the ratio of the non-polar solvent to the source of brown grease may range from about 10 v/v % to about 1000 v/v %. In various embodiments, the ratio of the non-polar solvent to the source of brown grease may range from about 10 v/v % to about 50 v/v %, from about 50 v/v % to about 250 v/v %, or from about 250 v/v % to about 1000 v/v %.

### (d) Optional Other Agents

Acids or bases may be added to the source of brown grease to adjust the pH of the mixture to minimize emulsion and facilitate the isolation of the lipid component. The acid(s) or base(s) may be added to the metal pipeline, the heating/separating tank, or both. Non-limiting examples of suitable acids or bases include sulfuric acid, hydrochloric acid, nitric acid, sodium hydroxide, and potassium hydroxide. In some embodiments, the source of brown grease may be adjusted to a pH that ranges from about 1 to about 9, from about 2 to about 8, or from about 3 to about 7. In one embodiment, the source of brown grease may be adjusted to a pH from about 6 to about 7.

### (e) Separating the Lipid Component Comprising Brown Grease

The process further comprises separating the lipid component from the aqueous and solid components in the source of brown grease, wherein the isolated lipid component comprises the reclaimed brown grease. Since the lipid component is less dense than the aqueous and solid components, the lipid component typically forms an upper layer that overlays the other components. A variety of means and/or equipment may be employed for separating the lipid component from the other components. For example, a separation tank may be utilized, wherein the upper lipid layer may be decanted and separated from other components. The separation tank may be vertical, horizontal, conical, or have any of a variety of shapes. Alternatively, the lipid component may be separated from the other components by using a centrifuge to remove the upper lipid layer. The heated source of brown grease may be pre-treated before transferred to a separation tank or centrifuge. For example, the heated source of brown grease may be filtered to remove particulate matter.

The lipid component may be removed from the heated source of brown grease while the heated source of brown grease is at the target temperature. Alternatively, the heated source of brown grease may be cooled to a slightly lower temperature before the lipid component is separated from the other components. In some embodiments, a heat exchanger may be used to recover some of the heat/energy from the heated source of brown grease during the separation step or from the heated components after the separation step. The heat exchanger may be stationary or mobile. In still another embodiment, the heated source of brown grease may be allowed to cool until the lipid phase solidifies, wherein the solidified layer of lipid comprising brown grease may be removed from the other components.

In embodiments in which the source of brown grease is heated in the presence of a non-polar solvent, the solvent may be removed from the lipid component after separation from the other components. The solvent may be removed by any of numerous methods well known to those of skill in the art. For example, the solvent may be removed under a vacuum (i.e., in vacuo), by evaporation, by distillation, and so forth. The solvent that is removed from the lipid component may be recovered, recycled, and reused. In one embodiment, the non-polar solvent may be a hexane and the hexane may be removed in vacuo from the lipid component comprising brown grease. In other embodiments in which the source of brown grease is heated in the presence of a non-polar solvent, the solvent may not be removed from the lipid component. For example, the non-polar solvent may be a vegetable oil or yellow grease and the lipid mixture comprising the brown grease and the vegetable oil or yellow grease may be used in downstream applications.

After removal of the lipid component comprising the brown grease, the aqueous component may be discharged into a wastewater disposal/treatment system or sewer system and the solid component may be disposed in a landfill site. The discharged aqueous and solid components also may be subjected to further chemical, physical, or biological processing before being disposed to sewer, wastewater treatment system, or landfill sites.

The aforementioned process may be repeated multiple times to increase the overall yield of brown grease isolated from the source of brown grease.

### (f) Optional De-Emulsification of the Separated Lipid Component

Sources of brown grease may contain significant amounts of FFAs, which have affinity towards water and, thus, may be emulsifying agents. As a consequence of the transfer through the pipeline and entry into the tank, the source of brown grease may have significant emulsion. The isolated lipid component comprising the brown grease, therefore, may be emulsified with water. The water content in the lipid component may be as high as 50 wt %. The presence of water in the emulsified lipid component may not cause any problems for some applications of brown grease, e.g., when it is used as a low-grade bunker fuel. However, it may be desirable to remove the residual water in the isolated lipid component for other applications, e.g., when the lipid component comprising brown grease is used as a raw material for biodiesel production.

In one embodiment, the lipid component may be optionally subjected to a centrifugation treatment for de-emulsifying the lipid component and separating the water from the de-emulsified lipid component. One skilled in the art will understand that various centrifugation speeds and duration times may be adopted for this process. Without limiting the scope of the invention, the centrifugation speed may range from about 500 rpm to about 100,000 rpm, from about 1,000 rpm to about 60,000 rpm, from about 2,000 rpm to about 20,000 rpm, or from about 5,000 rpm to about 12,000 rpm. After the centrifugation treatment, the water content in the isolated de-emulsified lipid component comprising brown grease may range from about 10 wt % to about 0.1 wt %, or from about 5 wt % to about 1 wt %.

In yet another embodiment, acidic water may be introduced into the centrifuge to wash the lipid component during the centrifugation treatment. The pH of the acidic water may range from less than about 1 to about 6, or from about 1 to about 4. The amount of the acidic water used for the centrifugation treatment may vary. It may range from about 1,000 wt % to about 1 wt %, from about 100 wt % to about 5 wt %, or from about 80 wt % to about 10 wt % of the lipid component. Typically, the acidic water is used to remove cationic salts like sodium chloride and/or trialkyl amines.

In another embodiment, a holding tank may be used to break the emulsions and remove residual water instead of a centrifuge. The lipid component is held inside the holding tank for a variable duration or until the emulsion breaks. Then, the top layer of lipid component comprising brown grease is separated from the aqueous layer.

### (f) Reclaimed Brown Grease

The lipid component comprising brown grease isolated from the source of brown grease typically comprises greater than about 15% FFAs. In various embodiments, the concentration of FFAs in the isolated brown grease may be about 20%, 25%, 30%, 35%, 40%, 45%, 50%, 55%, 60%, 65%, 70%, 75%, 80%, 85%, 90%, 95%, 99%, or 99.5%.

The isolated brown grease may be further treated chemically, physically, or biologically. As an example, a centrifugation step may be employed to remove residual water. Alternatively, the isolated brown grease may be washed with water and dried, or the isolated brown grease may be filtered to remove particulate matter. One skilled in the art will appreciate that numerous processes may be adopted for the further treatment of the isolated brown grease.

The isolated brown grease may find many applications in many industries. For instance, a combination of brown grease with other feeds can be used as feedstock for live animals. Isolated brown grease or a combination of brown grease with other materials like petroleum products can be used bunk oil (fuel). Moreover, isolated brown grease also may be further processed into jet fuel or other liquid fuels. Additionally, isolated brown grease may be used raw material for producing detergents, soaps, surfactants, cosmetics, anti-foaming agents, etc.

### (II) Process for Deodorizing a Grease Waste Mixture

Another aspect of the disclosure encompasses a process for decreasing the odor of a grease waste mixture. The method comprises contacting the grease waste mixture with a source of steam at a temperature from about 100° C. to about 160° C. and/or contacting the grease waste mixture with an oxidizing agent, wherein the odor of the grease waste mixture is substantially reduced.

In some embodiments, the grease waste mixture is contacted with both the source of steam and the oxidizing agent. Contact with the source of steam may occur before contact with the oxidizing agent, after contact with the oxidizing agent, or concurrently with the oxidizing agent. In other embodiments, the grease waste mixture is contacted with only the source of steam. In still further embodiments, the grease waste mixture is contacted with only the oxidizing agent. Those of skill in the art will appreciate that the various permutations of the process will depend upon the source of the grease waste mixture.

In general, the deodorized grease waste mixture will have a substantially reduced odor. That is, the concentration of volatile odorants may be reduced by at least 20%, at least 30%, at least 40%, at least 50%, at least 60%, at least 70%, at least 80%, at least 90%, at least 95%, or at least 99%.

### (a) Grease Waste Mixture

As used herein, the “grease waste mixture” may be grease trap waste or grease-containing wastewater, which are defined above in section (I)(a), or the lipid component comprising brown grease isolated from grease trap waste or grease-containing wastewater, as detailed above in section (I)(f).

(b) Contact with a Source of Steam

The process may comprise contacting the grease waste mixture with a source of steam at a temperature that ranges from about 100° C. to about 160° C., such that the odor of the grease waste mixture is substantially reduced. In certain embodiments, the temperature may range from about 100° C. to about 110° C., from about 110° C. to about 120° C., from about 120° C. to about 130° C., from about 130° C. to about 140° C., or from about 140° C. to about 160° C. In one embodiment, the temperature may be about 121° C. In another embodiment, the temperature may be about 134° C.

Without being bound to any particular theory, it is believed that contact with the source of steam may eliminate smell-generating microorganisms. Contact with the source of steam may also convert volatile compounds bearing mercapto groups into less volatile sulfur-containing compounds. Despite the mechanism, however, the odor of the steam-treated grease waste mixture is substantially reduced relative to that of the untreated grease waste mixture.

Contact with the source of steam may occur at a pressure from about 0 psi to about 35 psi. In some embodiments, the pressure may be about 5 psi, about 10 psi, about 15 psi, about 20 psi, about 25 psi, or about 30 psi.

The period of time that the grease waste mixture is contacted with the source of steam can and will vary. In general, the period of time may range from about 1 second to about 30 minutes. In various embodiments, the period of time may range from about 1 second to about 30 seconds, from about 30 seconds to about 3 minutes, from about 3 minutes to about 10 minutes, from about 10 minutes to about 20 minutes, or from about 20 minutes to about 30 minutes. In one embodiment, the temperature may be about 121° C., the pressure may be about 15 psi, and the period of time may be about 15 minutes. In another embodiment, the temperature may be about 134° C., the pressure may be about 30 psi, and the period time may be about 3 minutes.

Additionally, the grease waste mixture may undergo other physical treatments that may assist the steam treatment in odor reduction. For example, the grease waste mixture may be dry heat sterilized. Dry heat sterilization may be conducted at a temperature of about 180° C. for about 5 hours.

The steam treatment process may be repeated multiple times depending on the nature of the grease waste mixture.

(c) Contact with an Oxidizing Agent

The process also may comprise contacting the grease waste mixture with an oxidizing agent, wherein the odor of the grease waste mixture is substantially reduced. Contact with an oxidizing agent may convert volatile mercapto-containing compounds to non-volatile sulfur-containing compounds having a higher oxidation state (and a less offensive odor).

Non-limiting examples of suitable oxidizing agents include hydrogen peroxide, sodium carbonate peroxide, sodium dichloroisocyanurate, sodium hypochlorite, sodium perborate, sodium peroxide, and combinations thereof. In one embodiment, the oxidizing agent may be hydrogen peroxide.

The amount of oxidizing agent that is contacted with the grease waste mixture can and will vary. In general, the ratio of oxidizing agent to the grease waste mixture may range from about 10 wt/wt % to about 1000 wt/wt %. In some embodiments, the ratio of oxidizing agent to the grease waste mixture may range from about 10 wt/wt % to about 30 wt/wt %, from about 30 wt/wt % to about 100 wt/wt %, from about 100 wt/wt % to about 300 wt/wt %, or from about 300 wt/wt % to about 1000 wt/wt %.

In general, contact with the oxidizing agent occurs at a temperature ranging from about 25° C. to about 75° C. In some embodiment, the temperature of the contact may be about 25° C., about 30° C., about 35° C., about 40° C., about 45° C., about 50° C., about 55° C., about 60° C., about 65° C., about 70° C., or about 75° C.

In one embodiment, the oxidizing agent may be hydrogen peroxide, the ratio of hydrogen peroxide to the grease waste mixture may be about 1 wt/wt %, and contact between hydrogen peroxide and the grease waste mixture may be conducted at a temperature of about 25° C.

Contact with the oxidizing agent may be repeated multiple times depending upon the nature of the grease waste mixture.

### (III) Reducing the Levels of Sulfur and FFA in a Crude Biodiesel Solution

Yet another aspect of the invention provides a process of reducing the levels of sulfur and FFAs simultaneously in a solution of crude biodiesel, thereby forming a solution of biodiesel having reduced levels of sulfur and FFAs. Examples of sulfur-containing compounds and FFAs that may be present in the crude biodiesel solution are presented below:

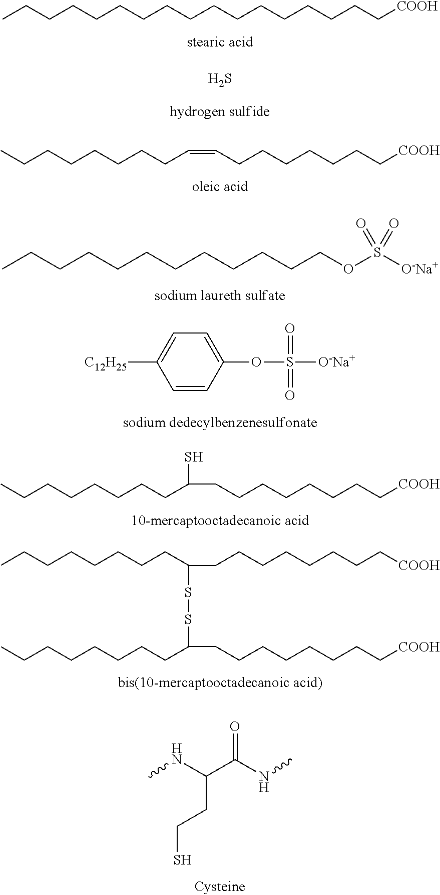

This process comprises contacting the solution of crude biodiesel with an insoluble base, wherein adducts of sulfur-insoluble base and FFA-insoluble base are formed. The process further comprises removing the adducts of sulfur-insoluble base and FFA-insoluble base to generate a solution of biodiesel having reduced levels of sulfur and FFAs.

### (a) Crude Biodiesel

A solution of crude biodiesel comprises alkyl monoesters of fatty acids, as well as FFAs, glycerides (i.e., tri-, di-, and mono-glycerides), glycerol, sulfur, sulfur-containing compounds, and other impurities.

(b) Contact with an Insoluble Base

A variety of insoluble bases may be used in the process of the invention. In general, the insoluble base will be a metal oxide, a metal hydroxide, a metal silicate, or a combination thereof. Non-limiting examples of suitable metal oxides include aluminum oxide (also called alumina), calcium oxide (also called lime, quicklime, burnt lime, or caustic lime), magnesium oxide (also called magnesia), nickel oxide, zinc oxide, copper oxide, derivatives thereof, and combinations thereof. Suitable metal hydroxides include, but are not limited to, aluminum hydroxide, aluminum oxide hydroxide, calcium hydroxide (also called hydrated lime, builders lime, slack lime, cal, slaked lime, or pickling lime), magnesium hydroxide, derivatives thereof, and combinations thereof. Suitable metal silicates include, but are not limited to, aluminum silicate (also called zeolite), calcium silicate, magnesium silicate, derivatives thereof, and combinations thereof. Alternatively, an aqueous solution containing an insoluble base may be used. The insoluble base has a solubility less than 10 g in 100 ml of anhydrous crude biodiesel, preferably less than 5 g in 100 ml of anhydrous crude biodiesel, more preferably less than 1 g in 100 ml of anhydrous crude biodiesel. However, such an insoluble base may be dissolved or suspended in an aqueous solution.

The insoluble base may also contain impurities and/or other components. For example, quick lime may contain silica, iron, alumina and magnesia. Such an insoluble base may be pre-treated chemically, physically or biologically before contacting it with the solution of crude biodiesel. For instance, quick lime may be pre-heated to a temperature above 100° C. to remove the molecular water, which may increase its activity towards FFAs and sulfonates.

The amount of the insoluble base that is contacted with the crude biodiesel solution can and will vary. In general, the ratio of the insoluble base to the solution of crude biodiesel will range from about 0.1 wt/wt % to about 30 wt/wt % of crude biodiesel. In various embodiments, the ratio of the insoluble base to the solution of crude biodiesel may range from about 0.1 wt/wt % to about 0.3 wt/wt %, from about 0.3 wt/wt % to about 1 wt/wt %, from about 1 wt/wt % to about 3 wt/wt %, from about 3 wt/wt % to about 10 wt/wt %, or from about 10 wt/wt % to about 30 wt/wt %.

Contact between the insoluble base and the crude biodiesel solution may occur at a temperature that ranges from about 25° C. to about 120° C. In some embodiment, the temperature of the contact may be about 30° C., about 40° C., about 50° C., about 60° C., about 70° C., about 80° C., about 90° C., about 100° C., about 110° C., or about 120° C.

The period of time during with the insoluble base is contacted with the solution of crude biodiesel may range from about 1 min to about 12 hours. In some embodiments, the contact time may range from about 1-5 min, from about 5-20 min, from about 20-60 min, from about 1-3 hours, from about 3-6 hours, or from about 6-12 hours. In one embodiment, the period of time during which the insoluble base is contacted with the solution of crude biodiesel may be about 1 hour.

During the period of contact, the insoluble base and the solution of crude biodiesel generally will be physically mixed by stirring, rotating, or any other appropriate means. Contact with the insoluble base leads to formation of sulfur-insoluble base adducts and FFA-insoluble base adducts.

In one embodiment, the insoluble base may be calcium oxide, and the ratio of calcium oxide to the solution of crude biodiesel may be about 1 wt/wt %. Contact between calcium oxide and the solution of crude biodiesel may occur at a temperature of about 60° C. for about 20-60 minutes.

### (c) Removing the Sulfur-Insoluble and FFA-Insoluble Base Adducts

The sulfur-insoluble base adducts and the FFA-insoluble base adducts may be removed from the solution of crude biodiesel by filtration, centrifugation, or combinations thereof. The insoluble base-treated solution of crude biodiesel may be passed through filter paper, a sand bed, a celite bed, and the like. The filtration may be conducted in conjunction with centrifugation. One skilled in the art will appreciate that conditions and methods to remove these adducts from crude biodiesel may vary depending on the nature of crude biodiesel.

The aforementioned insoluble base-treatment process may be repeated multiple times until the desired levels of FFAs or/and sulfur is reached. For example, the resultant biodiesel having reduced levels of sulfur and FFAs may comprise less than about 15 ppm of sulfur. Additionally, the resultant biodiesel having reduced levels of sulfur and FFAs may have an acid number of less than about 0.5 mg KOH/g.

(IV) Producing Biodiesel from a Lipid Mixture Comprising Brown Grease

Still another aspect of the disclosure encompasses a process for forming biodiesel from a lipid mixture comprising brown grease. The process comprises contacting the lipid mixture comprising brown grease with an alcohol in the presence of an esterification catalyst to form a crude biodiesel solution. The process further comprises contacting the crude biodiesel solution with an insoluble metal oxide, an insoluble metal hydroxide, an insoluble metal silicate, or combinations thereof to form the biodiesel solution.

### (a) Esterification Reaction to Form a First Solution

The process comprises contacting the lipid mixture comprising brown grease with an alcohol in the presence of an esterification catalyst to form a crude biodiesel solution. During this step of the process, FFAs in the lipid mixture comprising brown grease react with the alcohol to form fatty acid alkyl esters.

Lipid mixture comprising brown grease. The lipid mixture comprising brown grease may be isolated from a source of brown grease as detailed above in section (I). In some embodiments, the lipid mixture comprising brown grease may further comprise a non-polar solvent as detailed above in section (I)(c). In other embodiments, the lipid mixture comprising brown grease may further comprise other lipids derived from renewable materials. For example, the lipid mixture comprising brown grease may also comprise vegetable oils, animal fats, algal oils, food-based lipid wastes, industrial-based lipid wastes, and combinations thereof. Furthermore, the lipid mixture comprising brown grease or the source of brown grease from which it is derived may be deodorized as detailed above in section (II).

Alcohol. In general, any alcohol may be used in the esterification reaction. An alcohol refers to any compound having at least one hydroxyl group bound to a carbon atom of an alkyl or a substituted alkyl group. Thus, an alcohol may be linear, cyclic, or branched, and the hydrocarbyl moiety may be saturated or unsaturated. Alcohols suitable for use in this invention will generally have less than about 10 carbon atoms. In one embodiment, the alcohol may have from about 8 carbon atoms to about 10 carbon atoms. In another embodiment, the alcohol may have from about 5 carbon atoms to about 7 carbon atoms. In preferred embodiments, the alcohol may have from 1 carbon atom to about 4 carbon atoms. Suitable alcohols having from 1 carbon atom to about 4 carbon atoms include methanol, ethanol, propanol, isopropanol, butanol, and isobutanol. In one embodiment, the alcohol may be methanol. It should be noted that combinations of alcohols may also be used in the process of the invention.

The concentration of alcohol used in the esterification reaction can and will vary depending upon a variety of factors, including the nature of the lipid mixture comprising brown grease. The concentration of alcohol may range from about 1% to about 2000% by weight of the lipid mixture comprising brown grease. In one embodiment, the concentration of alcohol may range from about 1% to about 50% by weight of the lipid mixture comprising brown grease. In another embodiment, the concentration of alcohol may range from about 50% to about 100% by weight of the lipid mixture comprising brown grease. In an alternate embodiment, the concentration of alcohol may range from about 100% to about 500% by weight of the lipid mixture comprising brown grease. In still another embodiment, the concentration of alcohol may range from about 500% to about 1000% by weight of the lipid mixture comprising brown grease. In yet another embodiment, the concentration of alcohol may range from about 1000% to about 1500% by weight of the lipid mixture comprising brown grease. In another alternate embodiment, the concentration of alcohol may range from about 1500% to about 2000% by weight of the lipid mixture comprising brown grease.

Esterification catalyst. An esterification catalyst refers to a substance that starts or speeds up an esterification reaction. Some esterification catalysts may be dissolved in the substrates (i.e., the lipid mixture comprising brown grease and the alcohol). Non-limiting examples of such esterification catalysts include sulfuric acid, hydrochloric acid, titanium chloride, zirconium chloride and ferric chloride. Other esterification catalysts may have limited solubility in the substrates, such that the catalyst is essentially insoluble. Non-limiting examples suitable insoluble catalysts include sulfonated polymer resins (such as, e.g., like Dowex Marathon MSC or Dowex Monosphere 2030) or metals immobilized on a solid support. Insoluble catalysts may be recovered after the reaction, recycled, and reused.

Those skilled in the art will appreciate that the concentration of the catalyst used in the esterification reaction can and will vary, depending upon the nature of the lipid mixture, the temperature of the reaction, and so forth. In one embodiment, the concentration of the catalyst may range from about 0.1% to about 60% by weight of the lipid mixture comprising brown grease. In another embodiment, the concentration of the catalyst may range from about 0.5% to about 35% by weight of the lipid mixture comprising brown grease. In still another embodiment, the concentration of the catalyst may range from about 1% to about 20% by weight of the lipid mixture comprising brown grease.

Reaction conditions. The temperature at which the esterification reaction of the process is conducted may vary. In general, the temperature will be below the flash points of the substrates. Typically, the temperature of the reaction may range from about 25° C. to about 200° C. In various embodiments, the temperature of the reaction may range from about 40° C. to about 100° C. In alternate embodiments, the temperature of the reaction may be about 50° C., about 55° C., about 60° C., about 65° C., about 70° C., or about 75° C.

The duration of the esterification reaction can and will vary, depending upon the substrates and the temperature of the reaction. Typically, the duration of the reaction will be long enough for the reaction to go to completion, i.e., substantially all of the FFAs have been converted into fatty acid esters. Techniques well known in the art, such as gas chromatography (GC), nuclear magnetic resonance (NMR), or mass spectrometry (MS), may be used to determine the completeness of the reaction. In one embodiment, the duration of the reaction may range from about 5 seconds to about 48 hours. In another embodiment, the duration of the reaction may range from about 1 minute to about 24 hours. In still another embodiment, the duration of the reaction may range from about 5 minutes to about 12 hours. In an alternate embodiment, the duration of the reaction may range from about 10 minutes to about 6 hours. In yet another embodiment, the duration of the reaction may range from about 15 minutes to about 4 hours.

The pressure under which the reaction is conducted may vary. The pressure may range from low pressures, such as 40-60 kPa (˜6-9 psia) to high pressures, such as 350-1200 kPa (˜50-175 psia). Typically, however, the reaction may be carried out at atmospheric pressure, which is about 100 kPa (˜14.5 psia).

Typically, the reaction may be performed without an additional organic solvent; i.e., a solvent in addition to the alcohol substrate described above.

The esterification process may be conducted in a batch, a semi-continuous, or a continuous mode. The operations may be suitably carried out using a variety of apparatuses and processing techniques well known to those skilled in the art. Furthermore, some of the operations may be omitted or combined with other operations without departing from the scope of the present invention. In a preferred embodiment, the reaction may be performed in a continuous mode of operation. Accordingly, an insoluble catalyst may be packed in a catalyst bed for repeated uses in, for example, a continuous stirred tank reactor or in a plug-flow tubular reactor. The insoluble catalyst may be recovered, regenerated, and reused. The method used to recover the insoluble catalyst can and will vary, depending mainly upon the mode of operation of the reaction.

### (b) Reducing the Levels of Sulfur and FFA to Form the Biodiesel Solution

The process further comprises contacting the crude biodiesel solution formed in step (a) with an insoluble metal oxide, an insoluble metal hydroxide, an insoluble metal silicate, or combinations thereof to form the biodiesel solution. The biodiesel solution has reduced levels of sulfur and free fatty acids. Contact with the insoluble metal oxide, insoluble metal hydroxide, insoluble metal silicate, or combinations thereof is detailed above in section (III).

### (c) Optional Transesterification Reaction

The biodiesel solution having reduced levels of sulfur and FFAs may be further subjected to a transesterification reaction to form a biodiesel solution having reduced levels of glycerides. During the transesterification reaction, the glycerides in the biodiesel solution are reacted with an alcohol, in the presence of a transesterification catalyst, to form fatty acid alkyl esters. Suitable alcohols are detailed above in section (IV)(a).

Transesterification catalyst. A transesterification catalyst refers to a substance that starts, or speeds up a transesterification reaction. Non-limiting examples of transesterification catalysts include sodium hydroxide, potassium hydroxide, sodium methoxide, potassium methoxide, triethylamine, N-heterocyclic carbenes, and combinations thereof.

The concentration of the transesterification catalyst can and will vary depending upon the substrates, for example. In general, the concentration of the catalyst may range from about 0.1% to about 60% by weight of the solution of crude biodiesel. In another embodiment, the concentration of the catalyst may range from about 0.5% to about 35% by weight of the solution of crude biodiesel. In still another embodiment, the concentration of the catalyst may range from about 1% to about 20% by weight of the solution of crude biodiesel.

Reaction conditions. The temperature at which the transesterification reaction is conducted can and will vary, depending upon the substrates and the catalyst utilized. In one embodiment, the temperature of the reaction may range from about 15° C. to about 75° C. In an alternate embodiment, the temperature of the reaction may range from about 18° C. to about 40° C. In another embodiment, the temperature of the reaction may range from about 20° C. to about 30° C. In one embodiment, the temperature of the reaction may be at 25° C.

The duration of the transesterification reaction can and will vary, depending upon a variety of factors. Typically, the duration of the reaction will be long enough for the reaction to go to completion, i.e., all of the glycerides have been converted into fatty acid alkyl esters. Techniques well known in the art, such as gas chromatography (GC), nuclear magnetic resonance (NMR), or mass spectrometry (MS), may be used to determine the completeness of the reaction. In one embodiment, the duration of the reaction may range from about 5 seconds to about 48 hours. In another embodiment, the duration of the reaction may range from about 30 seconds to about 24 hours. In still another embodiment, the duration of the reaction may range from about 1 minute to about 3 hours. In an alternate embodiment, the duration of the reaction may range from about 5 minutes to about 2 hours.

The pressure under which the reaction is conducted may vary. The pressure may range from low pressures, such as 40-60 kPa (˜6-9 psia) to high pressures, such as 350-1200 kPa (˜50-175 psia). Typically, however, the reaction may be carried out at atmospheric pressure, which is about 100 kPa (˜14.5 psia).

The reaction will generally be carried out without an additional organic solvent; that is, a solvent in addition to the alcohol substrate.

The transesterification process may be conducted in a batch, a semi-continuous, or a continuous mode, as detailed above.

### (d) Final Biodiesel Solution

The reaction product (i.e., the biodiesel solution formed after step (b) or the biodiesel solution having reduced levels of glycerides) may be post-treated to remove reaction byproducts and/or impurities. In one embodiment, the post treatment process may include distillation to remove the alcohol from the biodiesel solution. In another embodiment, the post treatment process may include vacuum drying to remove water from the biodiesel solution. In yet another embodiment, the post treatment process may also include degumming to remove phosphatides or other solid residues from the biodiesel solution. In still another embodiment, the biodiesel solution may be washed with warm water to remove residual catalyst. In yet an alternate embodiment, the post treatment process may include the purification and fraction of fatty acid esters via distillation or vacuum distillation processes. One skilled in the art will know which process(es) to perform and how to perform them.

Typically, the final biodiesel solution will have a level of sulfur that is less than about 15 ppm. In some embodiments, the level of sulfur may be less than about 14, 13, 12, 11, 10, 9, 8, 7, 6, 5, 4, 3, 2, or 1 ppm. Additionally, the final biodiesel solution generally will have an acid number of less than about 0.5 mg KOH/g. In various embodiments, the acid number may be less than about 0.4, 0.3, 0.2, 0.1, 0.05, or 0.01 mg KOH/g.

The biodiesel solution formed by the processes of the present invention may be commercially useful for fuel compositions, lubricants, emulsifiers, plasticizers, intermediates for the production of products, such as soaps, detergents, or fragrances, and so forth. In an exemplary embodiment, the biodiesel solution may be used as a fuel composition. The fuel composition may be a biodiesel. Alternatively, the fuel composition may be a blend of a petroleum based diesel fuel and biodiesel.

### DEFINITIONS

To facilitate understanding of the invention, a number of terms and abbreviations, as used herein, are defined below:

The term “acyl” denotes a radical having the general formula RCO—, provided after the removal of a hydroxyl group from an organic acid. Examples of acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

The term “alkyl” embraces linear, cyclic, or branched hydrocarbon radicals having one carbon atom to about twenty carbon atoms. More preferred alkyl radicals are “lower alkyl” radicals having one carbon atom to about six carbon atoms. Examples of such radicals include methyl, ethyl, n-propyl, isopropyl, n-butyl, isobutyl, sec-butyl, tert-butyl, pentyl, iso-amyl, hexyl, and the like.

The term “biodiesel,” as used herein, refers to a composition of alkyl monoesters of fatty acids derived from a biological material, and which meets the specifications of ASTM D 6751.

The term “brown grease”, as used herein, is a generic term of a lipid whose FFA content exceeds 15 wt %. Brown grease may include trap grease, sewage grease, and black grease. It usually refers to lipids from grease traps or interceptors, sewage pipelines, and wastewater treatment or sewage facilities. It also includes lipids from other sources whose FFA content is above 15 wt %.

The term “crude biodiesel”, as used herein, refers to a composition of alkyl monoesters of fatty acids and other impurities such as FFAs, sulfur, phosphine, proteins, etc. Crude biodiesel does not meet the specifications of ASTM D 6751.

The terms “de-odor” or “deodorize”, as used herein, refer to the reduction of unpleasant smells or odors caused by volatile odorants.

As used herein, “esterification” refers to a chemical process of condensing fatty acids with an alcohol in the presence of a catalyst.

The term “fatty acid,” as used herein, refers to any of a large group of organic acids made up of molecules containing a carboxyl group (—COOH) at the end of a usually unbranched hydrocarbon chain. The hydrocarbon chain may have from about 4 to about 24 carbon atoms, or more specifically, from about 12 to about 22 carbons. The hydrocarbon chain may be saturated or unsaturated.

The term “free fatty acid” or “FFA,” as used herein, refers to the fatty acid product upon breakage of an ester link of a glyceride.

The term “glyceride,” as used herein, refers to esters formed from glycerol and fatty acids. Glycerol has three hydroxyl functional groups that may be esterified with one, two, or three fatty acids to form a mono-glyceride, a di-glyceride, or a tri-glyceride.

The term “grease trap waste”, as used herein, refers to all the materials collected from grease traps or interceptors, sewage pipelines, and wastewater treatment or sewage facilities.

The term “grease-containing wastewater” refers to wastewater that contains fats, oils, and grease.

As used herein, the terms “hydrocarbon” and “hydrocarbyl” describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties preferably comprise 1 to 20 carbon atoms.

The term “lipid”, as used herein, comprises FFAs and glycerides and is generally derived from a renewable material. The renewable material may be a vegetable oil, an animal fat, an algal oil, a food-based lipid waste, an industrial lipid waste, or a combination thereof. Non-limiting examples of suitable vegetable oils include artichoke oil, camelina oil, canola oil, castor oil, coconut oil, copra oil, corn oil, cottonseed oil, flaxseed oil, hemp oil, jatropha oil, jojoba oil, karanj oil, milk brush/pencil bush oil, mustard seed oil, neem oil, olive oil, palm oil, peanut oil, radish oil, rapeseed oil, rice bran oil, rubber seed oil, safflower oil, sesame oil, soybean oil, sunflower oil, and tung oil. Suitable animal fats include, but are not limited to, blubber, chicken fat, cod liver oil, fish oil, ghee, poultry fat, lard, and tallow. Suitable fish oils include anchovy oil, herring oil, lake trout oil, mackerel oil, menhaden oil, pollock oil, salmon oil, and sardine oil. Non-limiting examples of algal oils include those from Aphanizomenon flos-aquae, Bacilliarophy sp., Botryococcus braunii, Chlorophyceae sp., Crypthecodinium cohnii, Dunaliella tertiolecta, Euglena gracilis, Isochrysis galbana, Nannochloropsis salina, Nannochloris sp., Neochloris oleoabundans, Phaeodactylum tricornutum, Pleurochrysis carterae, Prymnesium parvum, Scenedesmus dimorphus, Schizochytrium sp., Spirulina sp., and Tetraselmis chui. Examples of food-based lipid waste include waste vegetable oil (WVO), spent frying oil, yellow grease, which is the reusable grease obtained from restaurant operations, and brown grease. Suitable examples of industrial lipid waste include deodorizer distillates and acid oils (soapstocks) generated as side streams during the production of oil and detergent products; tall oils, which are byproducts of the pulping of pinewood; and red oils from the candle industry. As will be appreciated by the skilled artisan, the lipid material may be a combination of materials derived from different sources. For example, the lipid material may be a combination of a vegetable oil and an animal fat.

The term “sewage”, as used herein, refers to all wastewater. “Sewage” includes domestic, municipal, or industrial liquid waste products disposed of, usually via a pipe or sewer or similar structure, or sometimes in a cesspool emptier.

The “substituted hydrocarbyl” moieties described herein are hydrocarbyl moieties that are substituted with at least one atom other than carbon, including moieties in which a carbon chain atom is substituted with a heteroatom such as nitrogen, oxygen, silicon, phosphorous, boron, sulfur, or a halogen (i.e., fluorine, chlorine, bromine, iodine) atom. These substituents also include carbocycle, aryl, heterocyclo, alkoxy, alkenoxy, alkynoxy, aryloxy, hydroxy, protected hydroxy, keto, acyl, acyloxy, nitro, amino, amido, nitro, cyano, thiol, ketals, acetals, esters, and ethers.

The term “transesterification,” as used herein, refers to the chemical process of exchanging the alkoxy group of an ester compound by another alcohol in the presence of a catalyst.

The term “wastewater”, as used herein, refers to any water that has been adversely affected in quality by anthropogenic influence. It comprises liquid waste discharged by domestic residences, commercial properties, industry, and/or agriculture and can encompass a wide range of potential contaminants and concentrations. In the most common usage, it refers to the municipal wastewater that contains a broad spectrum of contaminants resulting from the mixing of wastewaters from different sources.

### EXAMPLES

The following examples are given by way of illustration only and therefore should be constructed to limit the scope of the present invention.

All the chemicals were purchased from either Acros Organics (Somerville, N.J.) or Aldrich (Milwaukee, Wis.) and used as received without further purification. Grease trap waste was collected from grease hauling trucks, restaurant grease interceptors in Carbondale and the wastewater treatment plant in Marion, Ill. Other grease trap waste was a gift from restaurants in multiple locations: Atlanta, Ga.; Fort Lauderdale, Fla. and Hong Kong, China. ¹H NMR analyses were performed on a Varian VXR-300 system (Palo Alto, Calif.) with an Oxford wide-bore magnet, and the chemical shifts were reported in parts per million (ppm) downfield relative to tetramethylsilane using the residual proton resonance of solvents as the references (¹H NMR): CDCl₃ δ 7.27; CD₂Cl₂ δ 5.32 and (¹³C NMR): CDCl₃ δ 77.2; CD₂Cl₂ δ 54.0. Magnesol 600 R was purchased from Dallas Group of America Inc. (Whitehouse, N.J.) and used without further purification. The acid number tests were carried out by following ASTM D 664 and all other biodiesel tests were done by Midwest Laboratories (Omaha, Nebr.).

### Example 1

### Separation and Recovery of Brown Grease from Wastewater Using Hexanes

Grease trap waste (10 g) collected from a grease hauling truck was mixed with hexanes (50 mL) at 50° C. After 10 min, the hexane layer was separated from the rest of the materials using a separatory funnel. Hexanes were then removed in vacuo to yield a yellowish solid (2.1 g, 21%). ¹H NMR analyses showed that isolated brown grease comprised mainly (>95%) FFAs.

### Example 2

### Using a Lipid to Recover Brown Grease from Wastewater

Grease trap waste (10 g) was mixed with soybean oil (10 g) at 25° C. After 5 min, the soybean oil layer was separated from the other materials using a separatory funnel. A total weight of 11.8 g was obtained for the layer comprising brown grease and soy bean oil. ¹H NMR analyses showed that the mixture contained both FFAs and glycerides.

### Example 3

### Separation and Recovery of Brown Grease without Other Chemicals

About 100 g of grease trap waste was heated up to 80° C. and after 2 hour, the mixture was transferred into a separatory funnel. The top oil was separated and filtered through a filter paper (˜45 microns). After cooling to ambient temperature, the oil was solidified as a yellowish solid (25 g, 25%). ¹H NMR analyses showed that the majority (>95%) was FFAs.

### Example 4

### Steam Treatment of Recovered Brown Grease

Brown grease (10 g) recovered from grease trap waste was autoclaved at 121° C. for 15 min. A significant reduction of odors was found.

### Example 5

### Steam Treatment of Grease Trap Waste Followed by Recovery of Brown Grease

Grease trap waste (100 g) was autoclaved at 121° C. for 15 min. A significant reduction of odors was found. Then, the mixture was cooled to 60° C. and transferred to a separatory funnel. The hot oil was removed. After cooling to ambient temperature, the oil was solidified as a yellowish solid (23 g, 23%). ¹H NMR analyses showed that the majority (>95%) was free fatty acids.

### Example 6

### The Use of Calcium Oxide to Remove FFAs and Sulfur from Crude Biodiesel

After the removal of methanol, a solution of crude biodiesel had an acid number of 1.73 mg KOH/g and a total sulfur level of 298 ppm. Three parallel experiments using insoluble bases were executed:
(a) To 10 g of crude biodiesel was added 0.5 g (5 wt %) of quick lime (CaO). The mixture was stirred at 60° C. After 48 hours, the liquid biodiesel became a thick gel that could not be filtered. Presumably, the crude biodiesel polymerized.
(b) To 40 g of crude biodiesel was added 0.4 g (1 wt %) of quick lime (CaO). The mixture was stirred at 60° C. for 20 min. The mixture was divided into four equal portions (˜10 g) for testing different filtration media and centrifugation methods to remove the lime adducts:
   (i) Sand filtering bed (1 g of acid-washed sands): It took 8.43 min for the filtrate to pass through.
   (ii) Sand & celite bed (0.5 g acid-washed sands and 0.5 g celite): It took 7.71 min for the filtrate to pass through.
   (iii) Filter paper (average pore size: 50 micron): It took 31.74 min for the filtrate to pass through.
   (iv) Centrifugation (1,000 rpm, 1 min): there was a complete separation of the solid and filtrate.

The filtrate of each sample (i)-(iv) was analyzed. The acid numbers of all four samples were around 0.43 mg KOH/g and the total sulfur was 173 ppm. The sample [i.e., sample (i)] filtered through the sand bed (˜10 g) was treated with another 0.1 g (1 wt %) of quick lime (CaO). After 20 min at 60° C., the lime adducts were removed by using a sand filter bed (1 g). The acid number of the filtrate was brought down to 0.09 mg KOH/g and the total sulfur was 52 ppm.
(c) To 10 g crude biodiesel was added 0.5 g (5 wt %) of Magnesol 600R (MgO and MgSiO₃). The mixture was stirred at 60° C. for 20 min. The mixture was filtered out using a filter paper. The acid number of the filtrate was brought down to 1.05 mg KOH/g and the total sulfur was 288 ppm.

### Example 7

### Synthesis of ASTM Biodiesel from Grease Trap Waste

Brown grease extraction and de-odorization: Three liters of grease trap waste was treated with steam at 134° C. for 10 min. Then, the waste was cooled to ambient temperature and heated to 80° C. The mixture was filtered through a cheesecloth and the liquid was transferred to a separatory funnel where the top oil layer was separated. About 610 g of crude brown grease was obtained. Residual water in crude brown grease was removed via centrifugation (1,000 rpm, 20 min) at 60° C. 519 g of de-watered brown grease was recovered.

1^{st}-stage esterification reaction: De-watered brown grease (500 g) was mixed with 125 g methanol at 60° C. The mixture was circulated continuously through a catalyst column (length: 34 cm, diameter: 3 cm) comprising 75 g Dowex 50W. After 8 hours, over 66% of the FFAs in the brown grease were converted to methyl esters. Then, 250 g of water was added to the mixture and the oil layer was separated from the bottom aqueous methanol layer.

2^{nd}-stage esterification reaction: The recovered brown grease layer was mixed with another 125 g methanol. The mixture was heated to 60° C. and continuously circulated through the catalyst column for a second-stage esterification reaction. After additional 16 hours, over 97.9% of FFAs in the brown grease were converted into methyl esters. Then, 250 g water was added to the mixture and the top grease layer was separated easily from the aqueous methanol layer at the bottom.

Transesterification reaction: The aforementioned product after the second-stage esterification reaction was treated with 0.5 g NaOH in 125 g methanol. The mixture was stirred at 60° C. for 1 hour. Then, 250 g water was introduced and crude biodiesel was separated from the aqueous-methanol mixture in a separatory funnel.

Removal of sulfur and FFAs: The aforementioned crude biodiesel (497 g) after the transesterification reaction was treated with 1 wt % calcium oxide at 60° C. After 10 min, the solid was removed via centrifugation (1,000 rpm, 5 min). This calcium oxide step was repeated one more time. The oil was then mixed with 5 g zeolite and the mixture was stirred at 60° C. for 2 hours. Zeolite was removed via filtration and the resulted biodiesel was subjected to a series of additional filtration steps. Finally, 452 g pale-yellowish oil was obtained. Analyses done by Midwest Laboratories revealed that this biodiesel product met the specifications set in ASTM D 6751-09:

| | Level | | ASTM D6751 | |
|---|---|---|---|---|
| Analysis | Found | Status | Limits | |
| Cetane Number | 64.1 | Pass | 47 | minimum |
| Oxidation stability (hrs) | 3 | Pass | 3 | minimum |
| Flash point (° C.) | >150 | Pass | 130 | minimum |
| Water & Sediment (% volume) | <0.01 | Pass | 0.05 | maximum |
| Viscosity Kinematic (mm²/s) | 4.48 | Pass | 1.9-6.0 | |
| Sulfated Ash (% mass) | <0.01 | Pass | 0.02 | maximum |
| Sulfur (total) (ppm) | 13 | Pass | 15 | maximum |
| Copper Corrosion (3 hr 50° C.) | 1a | Pass | No. 3 | maximum |
| Cloud Point (° C.) | 16 | Pass | report | |
| Carbon Residue (% mass) | <0.02 | Pass | 0.05 | maximum |
| Acid Number (mg KOH/g) | <0.05 | Pass | 0.50 | maximum |
| Free Glycerine (% mass) | <0.001 | Pass | 0.020 | maximum |
| Total Glycerine (% mass) | 0.019 | Pass | 0.240 | |
| Boiling Point-Dist Temp (° C.) | 340 | Pass | 360 | maximum |
| Phosphorous (% mass) | <0.0001 | Pass | 0.001 | |
| Magnesium/Calcium (ppm) | <1.00 | Pass | 5 | maximum |
| Potassium/Sodium (ppm) | <4.00 | Pass | 5 | maximum |
| Biodiesel Visual Inspection | #1 | Pass | N/A | |
| Cold Soak Filtration (sec.) | 83 | Pass | 360 | maximum |

### Example 8

### Heating Grease Trap Waste Inside a Metal Tube Followed by Recovery of Brown Grease

About 3 kg of grease trap waste was pumped through a copper tube (101 H04) with an OD of 0.625″, wall of 0.121″, ID of 0.383″ and length of 10″. The outside of the copper tube was in direct contact with the flame from three Bunsen Burners. The mixture exiting from the tube was re-directed and pumped through the tube again. After 10 minutes, the grease trap waste was transferred to a separatory funnel, in which the top oil layer (˜650 grams) was removed. The crude oil was then subjected to centrifugation and 455 grams of brown grease was recovered. After cooling to ambient temperature, the oil was solidified as a yellowish solid. ¹H NMR analyses showed that the majority (>95%) was FFAs.

What is claimed is:

1. A process for deodorizing a grease waste mixture, the process comprising contacting the grease waste mixture with a source of steam at a temperature from about 100° C. to about 160° C., an oxidizing agent, or a combination thereof, such that the grease waste mixture has a substantially reduced odor.

2. The process of claim 1, wherein contact with the source of steam occurs at a pressure from about 0 psi to about 35 psi and a period of time from about 1 second to about 30 minutes.

3. The process of claim 1, wherein the oxidizing agent is chosen from hydrogen peroxide, sodium carbonate peroxide, sodium dichloroisocyanurate, sodium hypochlorite, sodium perborate, sodium peroxide, and combinations thereof; the ratio of oxidizing agent to the grease waste mixture is from about 10 wt/wt % to about 1000 wt/wt %; and contact with the oxidizing agent is conducted at a temperature from about 25° C. to about 75° C.

4. A method for simultaneously transporting and heating a source of brown grease, the method comprising passing the source of brown grease through a metal pipeline that is heated with a flame to a temperature from about 25° C. to about 95° C. such that the source of brown grease has a dynamic viscosity from about 1.002×10⁻³ NS/m² to about 3.15×10⁻⁴ NS/m².

5. A process for reclaiming brown grease from a source of brown grease, the process comprising:
a) passing the source of brown grease through a metal pipeline that is heated with a flame to a temperature from about 25° C. to about 95° C. such that the source of brown grease has a dynamic viscosity from about 1.002×10⁻³ NS/m² to about 3.15×10⁻⁴ NS/m², the source of brown grease comprising a lipid component, an aqueous component, and a solid component; and
b) separating the lipid component from the aqueous and solid components in the source of brown grease, wherein the separated lipid component comprises the reclaimed brown grease.

6. The process of claim 5, further comprising de-emulsifying the separated lipid component by centrifugation.

7. The process of claim 5, wherein the source of brown grease is pre-heated by a heat exchanger prior to introduction into the metal pipeline.

8. The process of claim 5, wherein the source of brown grease is screened or filtered prior to introduction into the metal pipeline.

9. The process of claim 5, wherein the pH of the source of brown grease is adjusted by introduction of an acid or a base.

10. The process of claim 5, wherein the separating occurs in the presence of a non-polar solvent chosen from an alkane, a substituted alkane, a cycloalkane, an acylglycerol, and combinations thereof.

11. The process of claim 5, wherein the separating occurs in a separation tank have a first discharge for the aqueous and solid components of the source of brown grease, and a second discharge for the lipid component comprising the reclaimed brown grease.

12. The process of claim 5, wherein the source of brown grease or the reclaimed brown grease is contacted with a source of steam at a temperature from about 100° C. to about 160° C., an oxidizing agent, or a combination thereof, such that the grease waste mixture or the reclaimed brown grease is substantially deodorized.

13. A process for reducing the levels of sulfur and free fatty acids in a crude biodiesel solution, the process comprising contacting the crude biodiesel solution with an insoluble base to form a treated biodiesel solution comprising at least one sulfur-insoluble base adduct and at least one free fatty acid-insoluble based adduct, and removing the sulfur-insoluble and free fatty acid-insoluble base adducts from the treated biodiesel solution to generate a biodiesel solution having reduced levels of sulfur and free fatty acids.

14. The process of claim 13, wherein the insoluble base is chosen from a metal oxide, a metal hydroxide, a metal silicate, and combinations thereof; the ratio of insoluble base to the crude biodiesel solution is from about 0.1 wt/wt % to about 30 wt/wt %; and contact with the insoluble base is conducted at a temperature from about 25° C. to about 120° C.

15. The process of claim 13, wherein the insoluble base is calcium oxide; the ratio of calcium oxide to the crude biodiesel solution is about 1 wt/wt %; contact between calcium oxide and the crude biodiesel solution occurs at a temperature of about 60° C.

16. The process of claim 15, wherein the biodiesel solution having reduced levels of sulfur and free fatty acids comprises less than about 15 ppm of sulfur and has an acid number of less than about 0.5 mg KOH/g.

17. A process for preparing a biodiesel solution from a lipid mixture comprising brown grease, the process comprising:
a) contacting the lipid mixture comprising brown grease with an alcohol in the presence of an esterification catalyst to form a crude biodiesel solution; and
b) contacting the crude biodiesel solution with an insoluble metal oxide, an insoluble metal hydroxide, an insoluble metal silicate, or a combination thereof to form a solution comprising at least one sulfur-insoluble base adduct and at least one free fatty acid-insoluble based adduct, and removing the sulfur-insoluble and free fatty acid-insoluble base adducts from the solution to generate the biodiesel solution.

18. The process of claim 17, wherein the biodiesel solution has reduced levels of sulfur and free fatty acids.

19. The process of claim 17, wherein the alcohol is a C1 to C10 alcohol or a combination thereof; the ratio of the alcohol to the lipid mixture comprising brown grease is from about 1 wt/wt % to about 2000 wt/wt %; the esterification catalyst is chosen from sulfuric acid, hydrochloric acid, titanium chloride, zirconium chloride, ferric chloride, a sulfonated polymer resin, and a metal immobilized on a solid support; the ratio of the esterification catalyst to the lipid mixture comprising brown grease is from about 0.1 wt/wt % to about 60 wt/wt %; and step (a) is conducted at a temperature from about 25° C. to about 200° C.

20. The process of claim 17, wherein the insoluble metal oxide is chosen from aluminum oxide, calcium oxide, magnesium oxide, derivatives thereof, and combinations thereof; the metal hydroxide is chosen from aluminum hydroxide, calcium hydroxide, magnesium hydroxide, derivatives thereof, and combinations thereof; the insoluble metal silicate is chosen from aluminum silicate, calcium silicate, magnesium silicate, derivatives thereof, and combinations thereof; the ratio of the insoluble metal oxide, insoluble metal hydroxide, insoluble metal silicate, or combination thereof to the crude biodiesel solution is from about 0.1 wt % to about 30 wt %; and step (b) is conducted at a temperature from about 25° C. to about 120° C.

21. The process of claim 17, wherein step (b) is repeated until the biodiesel solution comprises less than about 15 ppm of sulfur and has an acid number of less than about 0.5 mg KOH/g.

22. The process of claim 17, further comprising contacting the biodiesel solution with an alcohol in the presence of a transesterification catalyst to form a biodiesel solution having reduced levels of glycerides.

23. The process of claim 22, wherein the alcohol is a C1 to C10 alcohol or a combination thereof; the ratio of the alcohol to the biodiesel solution is from about 1 wt/wt % to about 2000 wt/wt %; the transesterification catalyst is chosen from sodium hydroxide, potassium hydroxide, sodium methoxide, potassium methoxide, triethylamine, and an N-heterocyclic carbine; the ratio of the transesterification catalyst to the biodiesel solution is from about 0.1 wt/wt % to about 60 wt/wt %; and contact occurs at a temperature from about 15° C. to about 75° C.
